(19) United States
(12) Patent Application Publication
(10) Pub. No.: US 2016/0271391 A1
(43) Pub. Date: Sep. 22, 2016

(54) TREATING AND DETECTING BIOLOGIC TARGETS SUCH AS INFECTIOUS DISEASES

(71) Applicant: Dragan Danilo Nebrigic, Austin, TX (US)

(72) Inventor: Dragan Danilo Nebrigic, Austin, TX (US)

(21) Appl. No.: 14/776,623

(22) PCT Filed: Mar. 14, 2014
(86) PCT No.: PCT/US14/29813

§ 371 Date: Sep. 14, 2015

Related U.S. Application Data
(60) Provisional application No. 61/786,128, filed on Mar. 14, 2013.
(60) Provisional application No. 61/880,161, filed on Sep. 19, 2013.

(51) Int. Cl. A61N 1/20; A61N 1/40; A61N 1/32; A61N 7/00; A61N 5/06; A61N 1/10; A61N 5/02

(52) U.S. Cl. CPC A61N 1/205; A61N 1/10; A61N 1/40; A61N 5/022; A61N 7/00; A61N 5/0624; A61N 1/32; A61N 2007/0004; A61N 2005/0661

(57) ABSTRACT

A non-pharmaceutical apparatus, system and process for treating and/or detecting biological targets, such as infectious diseases, are provided in various implementations. In one implementation, a medical device, system and/or process targets biologic targets such as microorganisms, fungi, bacteria and viruses resident in an infected environment. Further various method of detecting the biological targets are also provided.

### CROSS-REFERENCE TO RELATED APPLICATIONS

This application claims the benefit of U.S. provisional application No. 61/786,128, filed Mar. 14, 2013 and U.S. provisional patent application No. 61/880,161 filed on Sep. 19, 2013, each of which is hereby incorporated by reference as though fully set forth herein.

### BACKGROUND

Pharmaceutical drugs and ointments are the primary treatment modality for infectious diseases. Antibiotics, for example, are not immune to complications, side effects and over-use which leads to immune resistance in the population. As a result, a cost effective therapeutic option that does not cause immune resistance could potentially have widespread utilization across many infectious diseases.

In the case of nail fungus (onychomycosis), pharmaceutical oral medication such as Griseofulvin, Fluconazole are the medications of choice for treatment. These medications have a modest clinical efficacy rate of 10% as reported in clinical trials. New technologies are required as alternative treatment options to address this chronic problem. Phototherapy provided by lasers is the only device modality currently available. The phototherapy lasers use “bulk heat” as a mechanism of action. This procedure is painful since lasers are color sensitive, the treatment targets are not clear and focused, and visual detection for the treatment location is inaccurate. As a result, lasers currently have a limited application in the treatment of (onychomycosis) and other topical infections.

### SUMMARY

A non-pharmaceutical apparatus, system and process for treating and/or detecting biological targets, such as infectious diseases, are provided in various implementations.

In one implementation, a medical device, system and/or process targets biologic targets such as microorganisms, fungi, bacteria and viruses resident in an infected environment such as a surgical implant, nail bed, acne or a wound. In this implementation, the medical device, system and/or process can provide one or more (single or combination of modalities) of the following mechanisms of action for treating the infected environment:
(a) directly or indirectly electrically couples to physiological fluids in the infected environment to introduce ions into the fluid;
(b) migrate ions, including certain biologic targets, within the infected environment into a location where they can be more easily treated and/or detected;
(c) locally alter a pH of the infected environment (e.g., in situ) through an introduction or movement of ions to treat the biologic targets;
(d) use the introduced ions to reduce repulsive forces of the biologic targets within the physiological fluid and cause the targets to clot or precipitate within a region of the infected environment;
(e) once localized, treat the biologic targets in any manner more effectively. For example, in various implementations, the device can perform any combination of the following operations:
   (i) treat the biologic targets through bulk heating, a modulated signal, electro-acoustic energy and/or pH modification;
   (ii) use electrostatic, electro-acoustic, and/or electro-kinetic forces to treat a biologic target (e.g., use electrostatic, electro-acoustic, and/or electro-kinetic forces to compromise a cell wall of a biologic target compromise a cell wall of a biologic target);
   (iii) use ultrasound frequencies to cause motion of a complex target-external ion (e.g., a super-ion) and generate electrical current in the infected environment; and/or
   (iv) expose the localized biologic targets to broadband light or components of broadband light (e.g., UVA and/or UVB) that are effective in treating the biologic targets.

Each of the combinations may be performed such that the treatment is non-thermal, near non-thermal, non-dominant thermal, or includes therapeutic thermal heating depending on the particular application.

A medical device, system and/or process, in various implementations, can also detect the presence, absence and/or concentration of biologic targets within the infected environment to determine the effectiveness of the treatment and/or to determine if treatment is needed or desirable.

In one implementation, for example, a treatment device comprises an antenna including at least one electrode adapted to electrically couple with an environment comprising biological targets for delivering an electromagnetic signal to the environment. In one particular implementation, the electromagnetic signal comprises at least two frequency components, such as a relative low frequency component and a relative high frequency component. In various implementations, for example, the relative low frequency component comprises a frequency of less than about 1 MHZ (e.g., in the range from about 5 KHz to about 1 MHz) and the relative high frequency component comprises a frequency of greater than about 100 MHz, such as in the range from about 100 MHz to 220 GHz. Thus, the relative high frequency component may reside in RF and/or microwave frequency ranges depending upon application.

In this implementation, the relative low frequency component of the electromagnetic signal is selected to interact with the environment, such as by generating ions within a fluid of the environment comprising the biological targets. In one particular implementation, for example, the relative low frequency component is adapted to generate at least one electrical double layer within the fluid of the environment. The smaller wavelength relative high frequency component of the electromagnetic signal, however, allows targeting the relative low frequency component and its effects within the particular environment in which the biological targets are present.

The environment, for example, may include an organic and/or inorganic substrate, such as, but not limited to, tissue, bone, petri dish, surgical implant (e.g., metal (titanium, stainless steel, etc.) polymer), or other known substrates on or within which biological targets may exist. The biological targets, for example, may be disposed within a biological film (biofilm) that may include any group of microorganisms in which cells stick to each other on a surface. These adherent cells are frequently embedded within a self-produced matrix of extracellular polymeric substance (EPS). Biofilm extracellular polymeric substance, which is also referred to as a slime (although not everything described as a slime is a biofilm), is a polymeric conglomeration generally composed of extracellular DNA, proteins and polysaccharides. Biofilms may form on living or non-living surfaces and can be present, for example, in natural, industrial, hospital and other settings. In one implementation described herein a biofilm comprises a fluidic suspension located within or on a substrate of the environment.

Thus, various frequency components of the electromagnetic signal may be selected for desired interactions with the environment and/or the biological targets in the environment. The relative low frequency component, for example, may be selected to achieve one or more specific functionality such as described throughout this document. Functionalities, for example, may include generating ions and/or electrical double layers within, at or adjacent to the environment, controlling, moving or otherwise affecting one or more ion, particle, biological target, solution or other component, altering an environmental condition (e.g., pH), or the like.

Similarly, the relative high frequency component may be selected to direct the relative low frequency component (and its effects) within a discrete environment or target region, such as near the biological targets. Specifically, the relatively smaller wavelength of the relative high frequency component allows a more specific targeting of a region than the relatively longer wavelength of the relative low frequency component. In addition, one or both frequency components of the electromagnetic signal may be selected for a particular interaction with the environment and/or the biological target. In one particular implementation, for example, a relative high frequency component of the electromagnetic signal may be selected for its interaction with a particular substrate in the environment (e.g., a tissue) that may effectively demodulate the relative low frequency component (e.g., a modulation signal) from the selected relative high frequency component (e.g., carrier wave signal) within the target environment.

In one particular implementation, the coupling of the electromagnetic signal with the environment to target one or more biological target results in a non-thermal or near non-thermal treatment. For the purposes used herein, near non-thermal (or non-thermal therapeutic) refers to a condition where although incidental thermal heat is created by the interaction of the electromagnetic signal and the environment/biological targets, such as via parasitic heating, the level of thermal energy created falls short of creating a therapeutic effect with respect to the biological targets using the thermal energy created. Rather, the therapeutic treatment of the biological targets may be performed via one or more other mechanisms described herein. Similarly, any thermal effect may be a non-dominant thermal effect in which any therapeutic activity is not a dominant therapeutic effect of a treatment. In one implementation, for example, a non-dominant thermal effect may comprise at least an order of magnitude less than a dominant therapeutic effect of a treatment.

Further, coupling of the electromagnetic signal with the environment and/or biological targets may be performed up to a point where thermal heat is about to be created or up to a predetermined level of acceptable thermal heat and other methods of treatment may be used in addition to provide further treatment. In one implementation where a substrate disposed nearby the biological targets may be sensitive to thermal energy (e.g., bone and other heat sensitive tissues), a secondary, tertiary, etc. method of treatment may be introduced in combination with and/or in sequence with the coupling of the electromagnetic signal with the environment and/or biological targets.

In one particular implementation, for example, electroacoustic and/or opto-electrical interaction may be used in combination and/or in sequence with the use of the electromagnetic signal to treat the biological targets. In this manner, non-thermal or near non-thermal treatments can be performed that effectively treat the biological targets without creating a level of thermal energy that may damage a nearby substrate and/or cause a patient pain related to the thermal energy.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 shows an example environment in which biological targets are present.

FIG. 2 shows an example implementation in which an environment comprises a plurality of colloidal particles each having a positive charge or a negative charge.

FIG. 3 shows an example implementation in which an electrical double layer is formed within an environment via an application of an electromagnetic signal to a pair of electrodes and coupled to the environment.

FIG. 4 shows an example of a system for targeting nail fungus in a toe.

FIG. 5 shows an example implementation of a contact approach of a particle targeting device in which a pair of electrodes are arranged around a toe.

FIG. 6 shows an example implementation of a non-contact approach of a particle targeting device in which a pair of electrodes are arranged around a toe.

FIG. 7 shows an example schematic circuit diagram of an ultrasound pulser programmable logic device.

FIG. 8 shows an example of a sleeve antenna configured to slide over the distal end of a digit, such as the toe shown or a finger, for treating and/or detecting a biological target.

FIG. 9 shows an ear in which biological targets can be treated and/or detected as described herein.

FIG. 10 shows an example of a biological target microorganism in an electromagnetic field in which a plurality of generated ions surround the microorganism.

FIG. 11 shows an example of a biological target microorganism disposed in an ultrasound field in which the ultrasonic field is used to control and/or move the microorganism.

FIGS. 12A and 12B show an example of an ultrasound transducer crystal that can be used to generate an ultrasound field within an environment comprising biological targets.

FIG. 13 shows an example antenna including an RF matching network and an ultrasound matching network that can be used to couple an electromagnetic field and/or ultrasound field with an environment comprising biological targets.

FIG. 14 shows example disposable matching layers that may be used with an antenna, such as the ones shown in FIGS. 12 and 13, for coupling to the environment while reducing the likelihood that the antenna is contaminated by the biological targets.

FIG. 15 shows an example of a matching layer or waveguide dielectric comprising an RF inductive antenna coil and an ultrasound transducer that may be used for coupling an electromagnetic field and/or an ultrasound field with an environment comprising biological targets.

FIG. 16 shows an example of an antenna configured for inductively and capacitively coupling an electromagnetic radio frequency (RF) field and a transducer for coupling an ultrasound field with an environment comprising biological targets.

FIG. 17 shows another example of a focused multi-element antenna device for coupling an electromagnetic field and/or ultrasound filed with an environment comprising biological targets.

FIG. 18 shows a block diagram of example treatment and detection methodologies that may be used to treat and/or detect biological targets within an environment.

FIG. 19 shows an example graph of a zeta potential plotted with respect to a pH level that may be used in various implementations for detecting biological targets within an environment.

### DETAILED DESCRIPTION

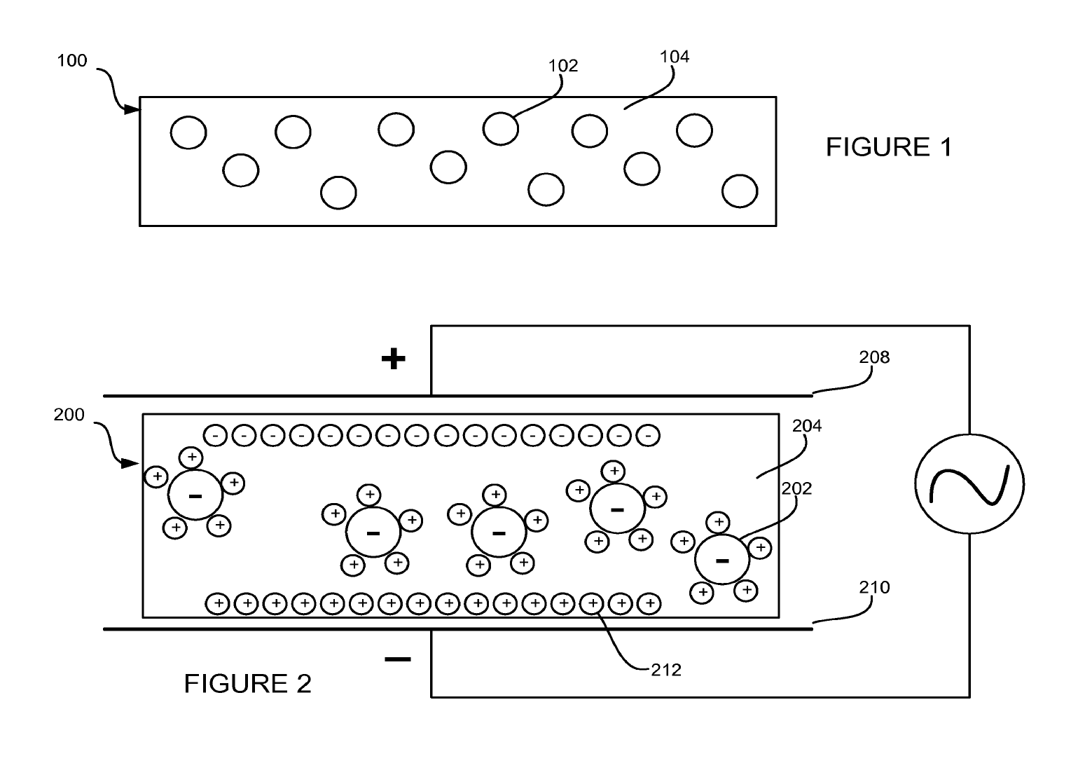

FIG. 1 shows an environment 100 in which biological targets 102 (e.g., fungi, bacteria, viruses or other microorganisms) are present. The biologic targets 102 for example, may be present within a biofilm or other configuration within the environment 100. The environment 100 may further comprise one or more substrates. In one particular implementation, the biological targets 102, for example, may be present as colloidal particles in a “suspension” of physiological fluids 104 (e.g., a viscoelastic fluidic suspension such as an interstitial fluid that flows between tissue cells 106 of humans and other animals). The biological targets may comprise charged or uncharged colloidal particles. A charged biological target 102, for example, may be positively charged or negatively charged. Bacteria, for example, are typically negatively charged particles. Fungi and viruses, for example, also have various charges.

FIG. 2 shows an example implementation in which an environment 200 comprises a plurality of colloidal particles 202 each having a positive charge or a negative charge. The plurality of colloidal particles 202, for example, may have invaded a tissue viscoelastic fluidic suspension 204. The suspension, for example, may be present in a physiological system, such as in a matrix of an infected nail, an infected wound, acne, or the like.

In the implementation shown in FIG. 2, a pair of electrodes 208 and 210 is provided adjacent to the environment 200 for electrically coupling with the environment 200. Although various implementations, such as this one, show a pair of electrodes configured for coupling with the environment antennas are also shown herein that comprise a monopole electrode configuration that may also be used in place of the dipole arrangement showed in this and other implementations. In one implementation, for example, a low frequency electromagnetic signal is applied across the pair of electrodes 208 and 210. In another implementation, an electromagnetic signal including a relative low frequency component (e.g., a modulation signal) and a relative high frequency component (e.g., like a carrier signal). Although a pair of electrodes are shown in this particular implementation, any number of electrodes may be used, including a single monopole electrode design as shown in some of the antennas discussed below. The electromagnetic signal, for example, may comprise an electromagnetic signal of less than about 500 KHz. In one particular implementation, for example, the electromagnetic signal may be in the range from about 5 KHz to about 200 KHz, although other frequencies are also possible.

In the implementation of a dual (or more component) electromagnetic signal a relative high frequency component (having a relative small wavelength) and the relative low frequency component (having a relative large wavelength), various frequency components of the electromagnetic signal may be selected for desired interactions with the environment and/or the biological targets in the environment. The relative low frequency component, for example, may be selected to achieve one or more specific functionality such as described throughout this document. Functionalities, for example, may include generating ions and/or electrical double layers within, at or adjacent to the environment, controlling, moving or otherwise affecting one or more ion, particle, biological target, solution or other component, altering an environmental condition (e.g., pH), or the like.

Similarly, the relative high frequency component may be selected to direct the relative low frequency component (and its effects) within a discrete environment or target region, such as near the biological targets. Specifically, the relatively smaller wavelength of the relative high frequency component allows a more specific targeting of a region than the relatively longer wavelength of the relative low frequency component. In addition, one or both frequency components of the electromagnetic signal may be selected for a particular interaction with the environment and/or the biological target. In one particular implementation, for example, a relative high frequency component of the electromagnetic signal may be selected for its interaction with a particular substrate in the environment (e.g., a tissue) that may effectively demodulate the relative low frequency component (e.g., a modulation signal) from the selected relative high frequency component (e.g., carrier wave signal) within the target environment.

The electromagnetic signal generates ions 212 in the environment 200. The ions 212 are free to migrate within the environment 200. In addition to the generated ions 212, intracellular and extracellular liquids contain ions 212 that are also free to migrate within the environment 200 (e.g., within an electric field generated by the electromagnetic signal applied to the electrodes 208 and 210). Where the electrode(s) 208 and 210 are in direct contact with a fluid 204 of the environment 200, ions may be introduced at a transition between the electrode(s) and the fluid 204 adjacent to the electrode(s) 208 and 210. Where the electrode(s) 208 and 210 are not in direct contact with the fluid 204, however, the ions can be introduced by an inductive—capacitive resonant charging process.

The electromagnetic field introduced by the electrodes 208 and 210 creates a “double layer” formed by the ions. Where the electrode(s) 208 and 210 are in direct contact with a fluid 204 of the environment (e.g., through a porous barrier such as a nail), the double layer is created at the transition between the electrode(s) 208 and 210. In the double layer, a surface charge of the electrode(s) 208 and 210 is mirrored by a parallel layer of ions within the fluid 204. The ions in the fluid form a diffuse layer of free ions under the influence of electric attraction and thermal motion.

Where the electrodes are not in contact with a fluid of the environment 200, however, the double layer may be created at a transition in which different layers or objects have different material or electrical properties. A double layer may similarly form at various tissue transitional surfaces (e.g., a nail, a nail matrix or other tissue transition). Thus, a layer of ions within a fluid 204 may mirror a parallel surface charge on a tissue within the environment 200.

Ions generated in the environment 200 are also attracted to and surround the charged colloidal particles 202. The ions, for example, may alter a pH of the environment 200 and/or alter a charge of individual colloidal particles 202 within the environment 200. Many biological targets are sensitive to pH and, thus, by creating ions (e.g., hydrogen or hydroxide ions) in the environment the pH within the environment (or within a closely controlled region of the environment) may be controlled to create an environment inhospitable to a particular type of target particle. Depending upon the target, pH can be controlled in situ to provide an inhospitable environment for the target. Thus, a pH of an environment may be controlled to be more acidic or basic depending on a particular target.

In addition, a charge of the colloidal particles 202 (e.g., proteins, bacteria, and fungus) within a suspension provides for reciprocal repulsion of the particles that keeps those particles in suspension. A loss of charge, however, can reduce the repulsive forces of the colloidal particles 202 (e.g., biological targets) which, in turn, can lead to clotting and precipitation of the particles within the physiological fluid.

Charged particles (e.g., charged colloidal or other charged target particles) within the environment can also be detected, controlled (e.g., oriented or displaced), and/or treated through an electrical coupling (e.g., capacitive or inductive coupling) and/or electroacoustics via the electrodes 208 and 210 (or another set of electrodes or antennas). A signal applied to the pair of electrodes 208 and 210, for example, can be used to couple the electrodes 208 and 210 to environment 200 to provide an electrostatic or electromagnetic charge in the environment 200 in which the targets such as the charged colloidal particles 202 reside.

In one particular implementation in which a double layer is formed within the environment 200, for example, the double layer can be used to localize the targets such as charged colloidal particles 202 in a particular region of the environment. The particles 202 localized within region, for example, may be easier to treat by virtue of the region in which they are localized. Nail fungus targets, for example, may be able to be localized within a nail bed under a nail and away from a root of the nail so that they may be more easily treated (e.g., via a laser or microwave bulk heating approach). In addition, the localized particles 202, may also be more effectively treated simply by their proximity to each other (e.g., a given treatment may be more effective since the particles 202 are localized together for treatment and a higher percentage of the targets 202 may be treated with the same treatment technique).

Coagulated target particles, for example, can be targeted for treatment, such as with energy to heat the targeted particles (e.g., bulk heating), a modulated signal (e.g., an amplitude modulated radio frequency signal superimposed on a “charging” direct current signal), electroacoustic energy, or any other targeted treatment methodology. As described above, for example, a pH of the environment 200 can controlled creating ions within the environment (e.g., within a specific region of the environment). In addition, positive ions of a target particle may be “pulled off” the target particle so that the particle will not spread and can be destroyed. Once damaged and/or isolated, a target particle may be destroyed through bulk heating, pH manipulation, electroacoustic energy, optoelectric treatment such as broadband light or effective components of broadband light (e.g., UVA and/or UVB) and/or through other methodologies. In one implementation, for example, electrostatic, electroacoustic, and/or electrokinetic forces may be used to compromise a cellular wall of a target particle.

In another implementation, the environment 200 is charged using electrostatic energy and/or the modulated signal described above, and a specific ultrasound frequency signal is also applied to the environment 200. The ultrasound frequency signal may be applied to the environment 200 via the pair of electrodes 208 and 210 or via another source. The ultrasound frequency causes motion of a complex “target-external ion” (e.g., a super-ion) and electrical current is generated in the environment 200 due to mechanical motion of the target-external ion. The generated electrical current, in turn, can provide a local voltage breakdown between the target-external ions other close target-external ions to create mechanical destruction of the target particles (e.g., destruction of a target fungus stem at a location where the fungus is tethered, comprising a cellular wall of a target particle, or the like). In this particular implementation, heating caused by the electroacoustic generated current can further affect free particles within the environment 100 (e.g., free fungus spores that have broken off the target fungus stems).

Electrically “presoaked” tissue with immobilized colloidal particles (biological targets) can be further treated with non-focused or partially focused (non-point HIFU) energy ultrasound energy. In one implementation transducer frequency delivered depends on a depth and target treatment location. In a nail treatment implementation, for example, application of ultrasound energy may be delivered at a frequency between 7 and 14 MHz or for other skin application between 2.2 MHz and 14 MHz. One skilled in the art, based on this disclosure, however, would readily appreciate that these are merely example implementations recognize that other frequency ranges may be used for these particular or many other applications.

In one implementation, ultrasound introduces acoustic energy that titrates a colloidal particle-charge system and moving charge in an acoustic field created local eddy currents that locally create cell wall heating and thermal breakdown locally. Also, a colloidal particle-charge system may be vibrated at an ultrasound frequency to bring colloidal particles beyond an elastic cell wall barrier to mechanically compromise a biologic target to damage or destroy microorganisms.

A secondary effect of ultrasound excitation is a cavitation effect caused by strong shear fields. For example, microorganism in some cases are sufficiently long (e.g., fungus) that they could break under a strain induced in an ultrasound field. Ultrasound effects can also be carefully tuned via a transducer frequency design to have a minimal thermal component and/or a non-dominant thermal component of a treatment (i.e., thermal energy does not comprise a dominant modality of treating a biological target), rather although thermal energy may be created, other modalities described herein comprise the dominant manner of treatment.

In one particular implementation, for example, an ultrasound effect may be increased or maximized by connecting a firing sequence of an ultrasound transducer to a firing sequence of a modulated electromagnetic energy source (e.g., antenna) due to the formation of an electric double layer and a parasitic discharge. Although several ultrasound transducers and electromagnetic antenna designs are described herein, many other designs may also be useful in one or more of the treatments or devices described herein as well.

Electroacoustic phenomena arise when ultrasound radiation propagates through a fluid containing ions. The phenomena moves the ions, and the motion generates electrical signals because the ions have an electric charge. The coupling between ultrasound and an electric field is referred to as electroacoustic phenomena. Fluid, for example, may comprise a simple Newtonian liquid, or a complex heterogeneous dispersion, emulsion or even a porous body. Examples of electroacoustic effects, depending on the nature of the fluid, include the following: (i) Ion Vibration Current/Potential (IVI) in which an electrical signal arises when an acoustic wave propagates through a homogenous fluid; (ii) Streaming Vibration Current/Potential (SVI) in which an electric signal arises when an acoustic wave propagates through a porous body in which the pores are filled with fluid; (iii) Collowid Vibration Current/Potential (CVI) in which an electric signal arises when ultrasound propagates through a heterogeneous fluid, such as a dispersion or emulsion; and (iv) Electric Sonic Amplitude (ESA), an inverse of a CVI effect, in which an acoustic field arises when an electric field propoagates through a heterogeneous fluid.

The movement of ions (including free ions and/or colloidal particles) through a fluid, such as in an environment comprising biological targets, creates a current.

A collaborative action between electrostatically or an amplitude modulated antenna induced electric double layer and acoustic waves introduced by an ultrasound source provides a treatment (e.g., destruction, reduction, damage, injuring, or other treatment) for biological targets within an environment. In one implementation, for example, an electric double layer can be regarded as behaving like a parallel plate capacitor with a compressible dielectric filling. Compressing the dielectric filling of a specific kind (e.g., size, dynamics, etc.) could be used as an identification of the colloidal particles/biologic target organisms (e.g., biologic target microorganisms). In this implementation, electrical noise detection can also be used to detect a presence of a certain size target, activity, kill rate and the like.

Use of an acoustic wave source to create a streaming vibration current representing an electrical signal that arises when an acoustic wave propagates through a porous body of the environment (e.g., a nail plate or other porous body) in which the pores are filled with a fluid that couples as a mediator between the porous body and an ultrasound electrode. The same effect could also be enhanced by an electrostatic approach as described above, a galvanic electrolysis and/or an AC modulation approach.

In yet another implementation, target particles 202 may be exposed to broadband light or to one or more components of broadband light, such as UVA and/or UVB to destroy or otherwise harm the target particles 202. Where the target particles 202 have been localized, for example, broadband light may be directed onto the localized target particles 202. Similarly, where motion of the target particles 202 is controlled, broadband light may be directed into a path through which the target particles 202 will move to destroy or otherwise harm the target particles 202. In addition, where target particles 202 are free floating in the environment 200 (e.g., spores broken off stems of fungus), the free floating particles 202 may be exposed to broadband light within the environment to destroy or otherwise harm the target particles 202. Where a fungus spore has broken off of stems of the fungus, for example, exposure of the spores to broadband light may prevent the spore from starting another stem root at the other location as well where the light could affect any superficial fungus, such as a surface of a nail plate or a mission nail plate location.

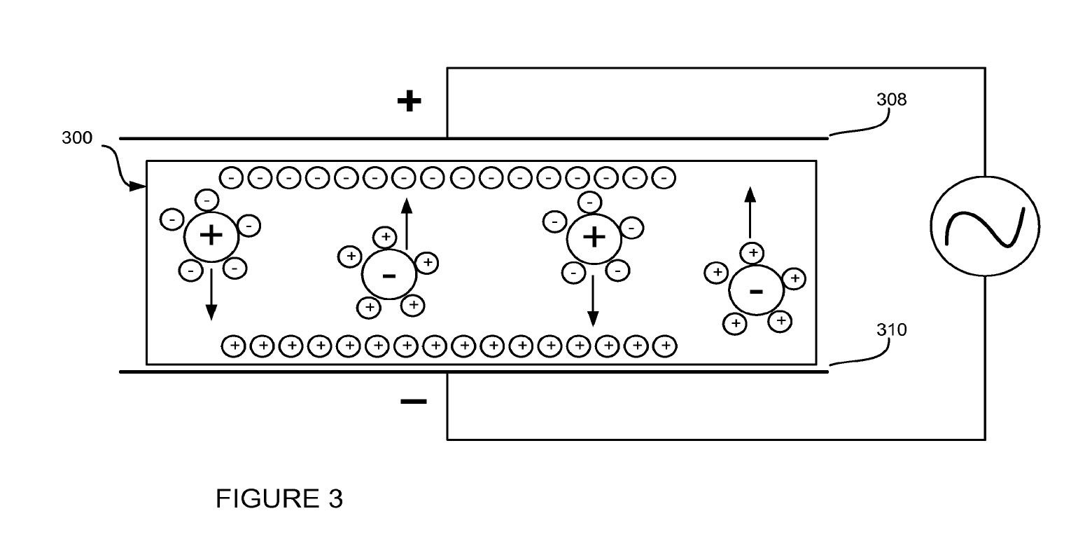

FIG. 3 shows an example implementation in which an electrical double layer is formed within an environment 300 via an application of an electromagnetic signal to a pair of electrodes 308 and 310 coupled to the environment. In this implementation, the electrical double layer is created proximal to a transitional surface in or adjacent to the environment 300. A transitional tissue within the environment 300, for example, is electrically charged and ions within the environment surround one or more target particles within the environment to electrically charge the particles to an isoelectric point. At an isoelectric point, a colloidal system is least stable from a zeta potential standpoint. The isoelectric point is related to a specific pH value for which the zeta potential is equal to 0 mV.

Clustering groups of combined particles (e.g., target particles and surrounding ions) to larger groups of particles creates gradients of materials with a more substantial electrical capacitive difference relative to a surrounding tissue (i.e., colloidal islands). The colloidal islands can then be exposed to higher frequencies (e.g., between 1 MHz to 2.4 GHz or similar frequencies) where Maxwell-Wagner conditions are dominant by selectively heating the colloidal islands alone. In addition, the colloidal islands can have pH values modified locally for a short time period to a pH value(s) that provide unfavorable living conditions for a particular living target particle. At the same time, the colloidal islands can be exposed to electrical force based vibration where the particle walls are compromised to destroy or damage the target particles within the colloidal islands.

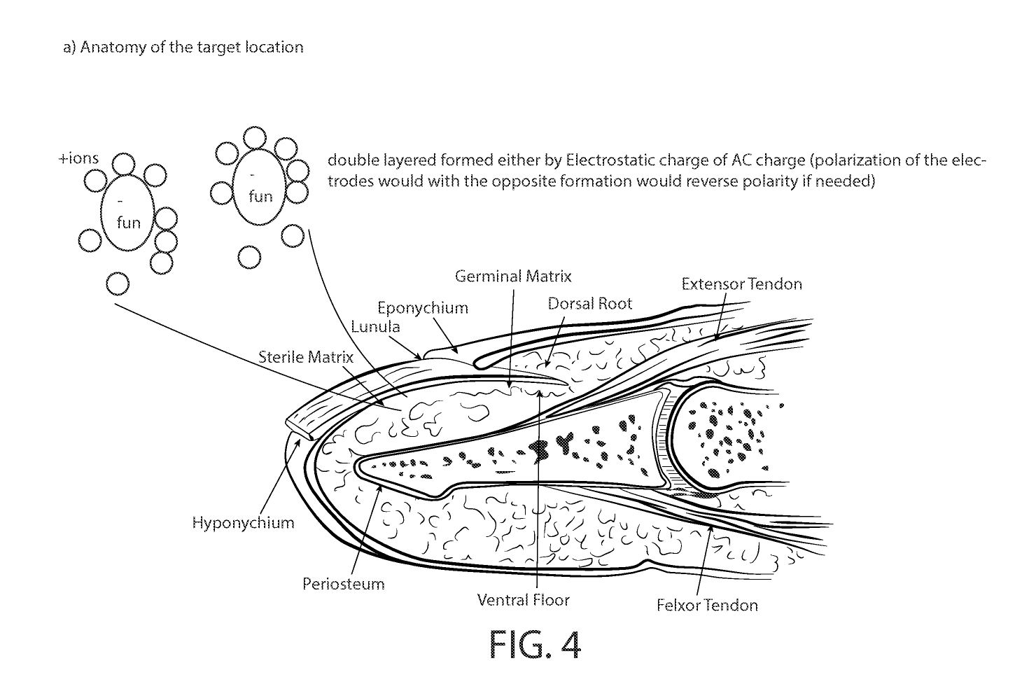

FIG. 4 shows an example of a system for targeting nail fungus in a toe. The system comprises a pair of electrodes that forms a double layer under a toenail. In the implementation shown in FIG. 4, a target particle comprises a negatively charged fungus particle, although any other target particle having a positive or negative charge may be used. Where a positive charged target particle is used, for example, the polarization of the electrodes may be reversed to reverse the charge of the double layer within the toenail environment.

The double layer, for example, may be formed by an electrostatic charge and/or by an alternating current (AC) charge. The alternating current charge, for example, may comprise an amplitude modulated radio frequency superimposed on an intermittent charging direct current signal.

In this particular implementation, the double layer comprises positively charged ions that are attracted to and interact with the negatively charged fungus target particles. As shown in FIG. 4, the fungus target particles are each surrounded by a plurality of positively charged ions generated by the electrostatic charge and/or alternating current charge. As described above with respect to FIG. 1, the double layer field may be used to move, align, or locate the fungus target particles to aid treatment of the target particles. Further, the positive ions increase the ionization of the environment in which the toe is exposed.

The increased ionization may be used to alter a pH of the toenail environment to increase the hostility of the toenail environment to the targeted fungus particles or to otherwise increase an effectiveness of the treatment of the targeted fungus. As described above, the increased ionization of the environment can decrease a charge of target particles within a suspension to reduce repulsion between the particles and encourage clotting and precipitation of the target particles. Once the target particles are coagulated within the sterile or germinal matrix of the toe, the target particles may be treated in any number of ways, such as by breaking down the targeted particles (e.g., with electrostatic or AC energy), exposure to electromagnetic vibration to compromise a cellular structure of the targeted particles, electroacoustic current generation created by motion of ionic targets in suspension (e.g., a physiological fluid such as a interstitial fluid), a current induced cellular wall breakdown, application of broadband light, bulk heating, and/or the like.

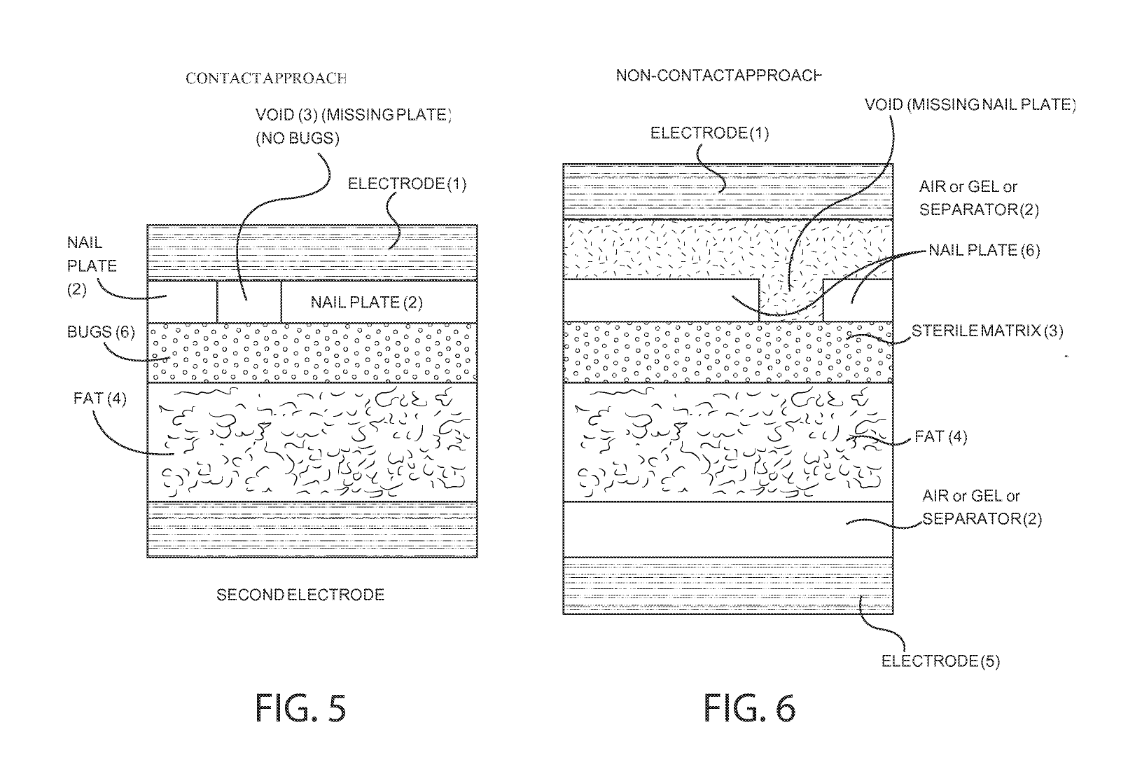

FIG. 5 (labeled “Contact Approach”) shows an example implementation of a contact approach of a particle targeting device in which a pair of electrodes are arranged around a toe. In this particular implementation, a first electrode is positioned directly adjacent to a nail plate of a patients toenail. The second electrode is electronically coupled to the first electrode and is arranged to provide an electrical field within a patient, such as a sterile matrix and/or a germinal matrix of a nail bed of a toenail. In FIG. 5, the nail plate is shown having a void where the toenail had fallen off due to a fungus infection of the toenail.

FIG. 6 (labeled “Non-Contact Approach”) shows an example implementation of a non-contact approach of a particle targeting device in which a pair of electrodes are arranged around a toe. In this particular implementation, a first electrode is separated from the nail plate of the patient by a layer of air, gel, or other separator. The second electrode is electronically coupled to the first electrode and is arranged to provide an electrical field within a patient, such as a sterile matrix and/or a germinal matrix of a nail bed of a toenail, through the layer of air, gel, or other separator material.

In FIG. 6, the nail plate is shown having a void where the toenail had fallen off due to a fungus infection of the toenail. In this particular implementation, the conductive gel or other separator may extend into the void to make a better connection with the sterile and /or germinal matrix of the toenail.

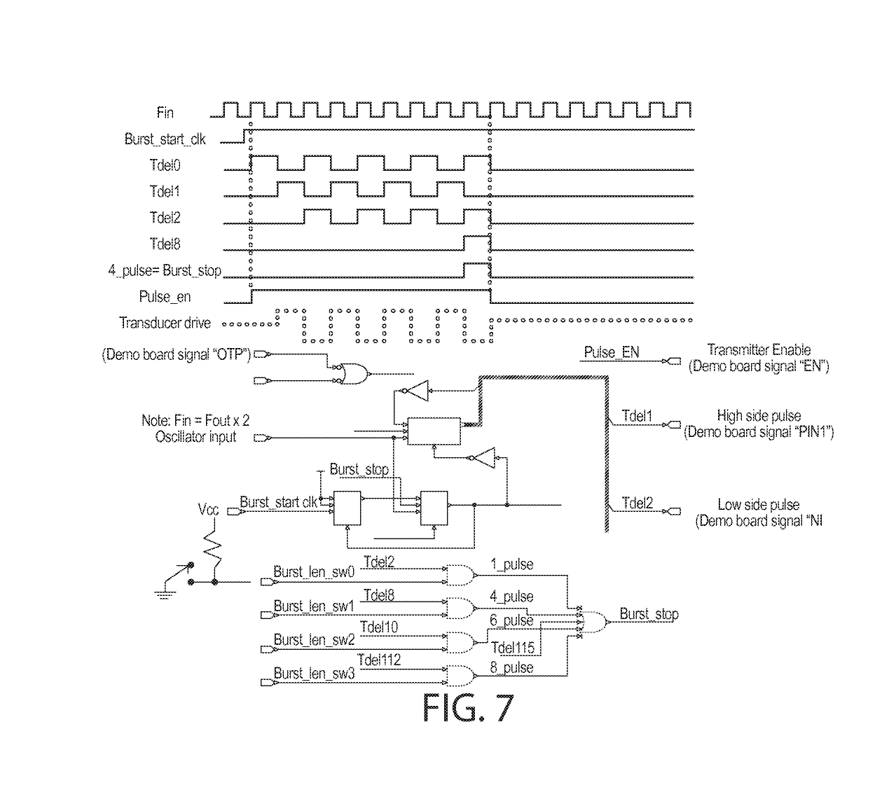

FIG. 7 illustrates an example schematic circuit diagram of an ultrasound pulser programmable logic device that may be used to provide an ultrasound signal for coupling with an environment comprising biological targets.

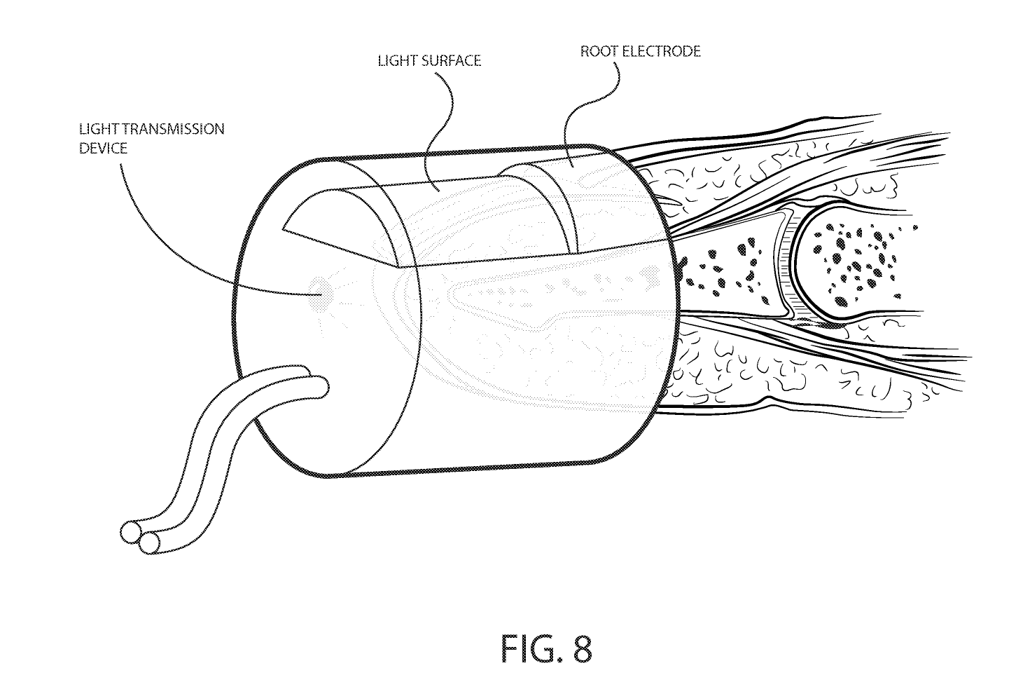

FIG. 8 shows an example of a sleeve antenna configured to slide over the distal end of a digit, such as the toe shown or a finger, for treating and/or detecting a biological target. In this particular implementation, for example, the sleeve antenna comprises a root electrode for introducing an electromagnetic field to the underlying foot of the nail and also a light transmission device (e.g., fiber optics, optical waveguide or the like). In addition to applying an electrostatic field through the root electrode, light transmission of broadband light (or one or more component thereof) can be applied to the environment (in this case a nail bed of the underlying toe). In this manner, both an electrostatic and an opto-electrical mechanism of action are provided by providing light through the light transmission element.

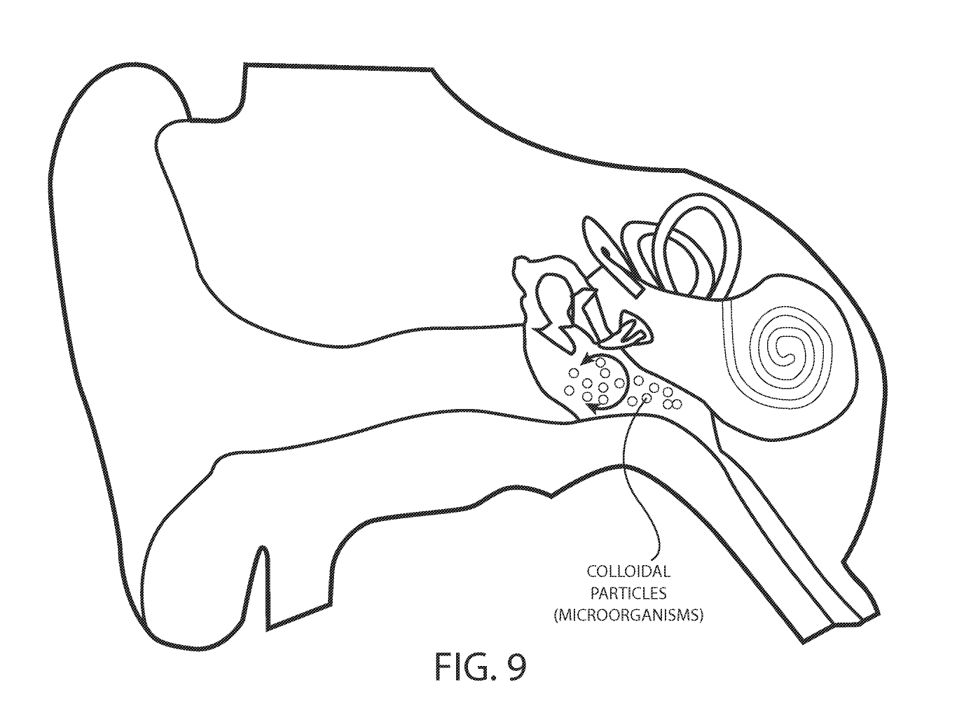

FIG. 9 shows an ear in which biological targets can be treated and/or detected as described herein. An ear infection behind an ear drum, for example may include colloidal particles in solution behind the ear drum.

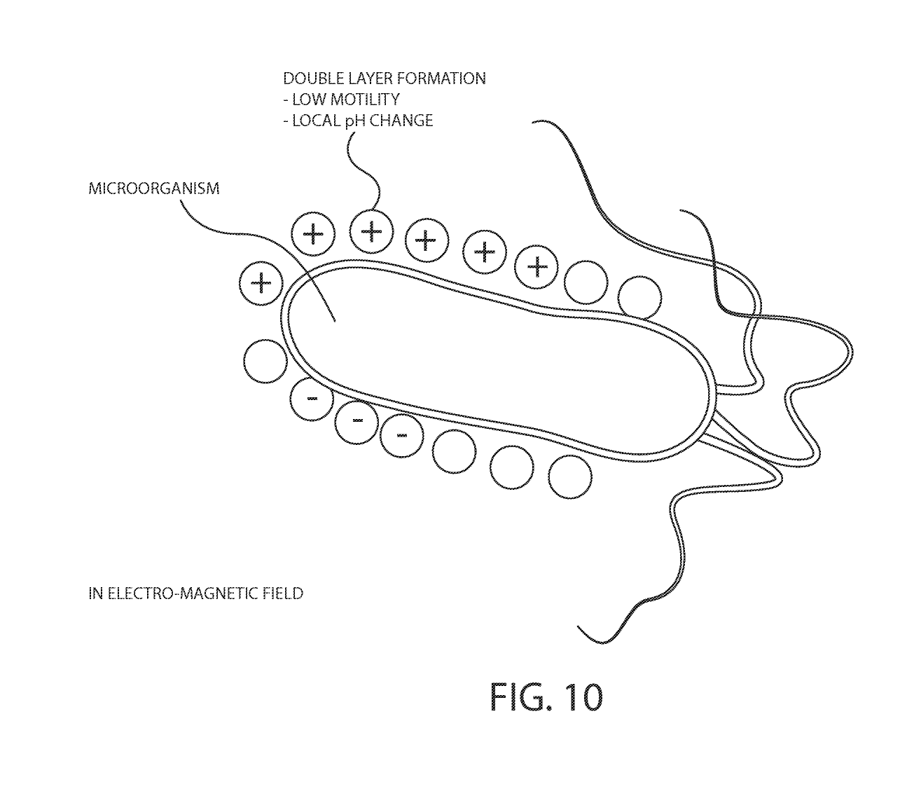

FIG. 10 shows an example of a biological target microorganism in an electromagnetic field in which a plurality of generated ions surround the microorganism. The ions, in this implementation, form an electrical double layer around the biological target and also provide a local pH change in situ. The double layer formation around the target as well as the change in pH can decrease the motility of the microorganism.

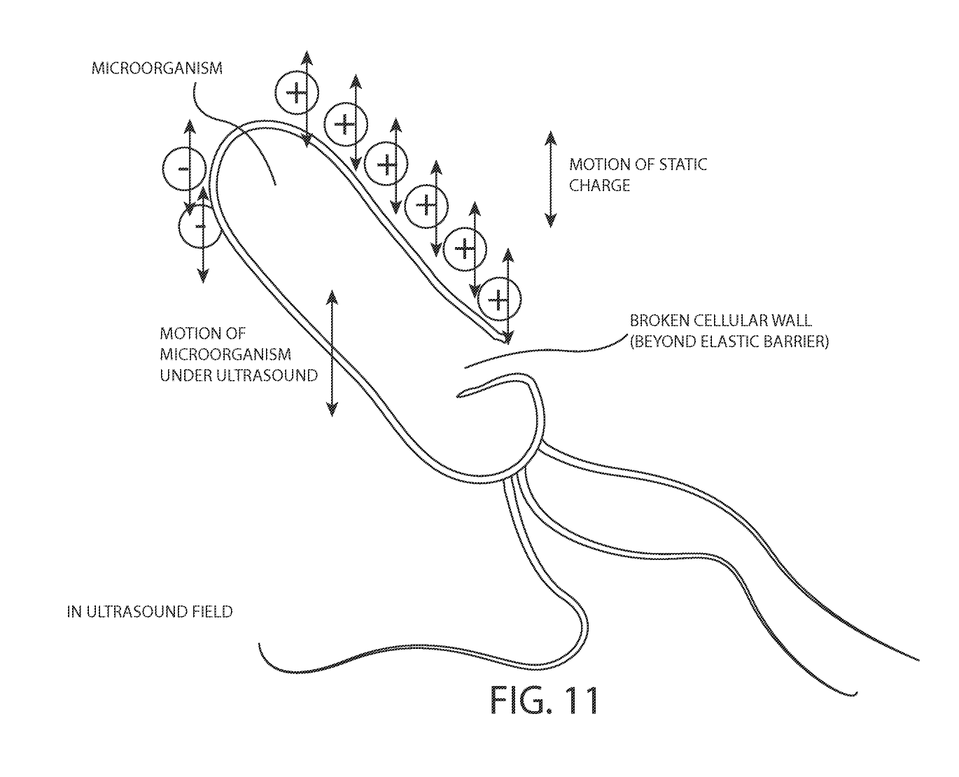

FIG. 11 shows an example of a biological target microorganism disposed in an ultrasound field in which the ultrasonic field is used to control and/or move the microorganism. For example, the ultrasound field may be used to provide motion and moving the microorganism with it. In addition, the motion, in various implementations, may be used to break down a cellular wall beyond an elastic bather of the target microorganism.

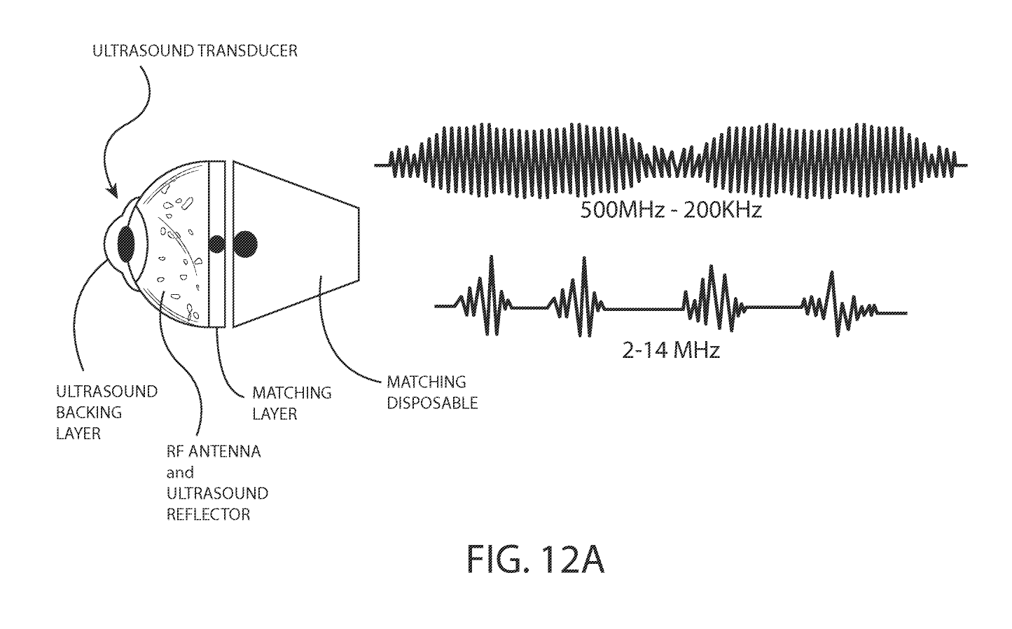

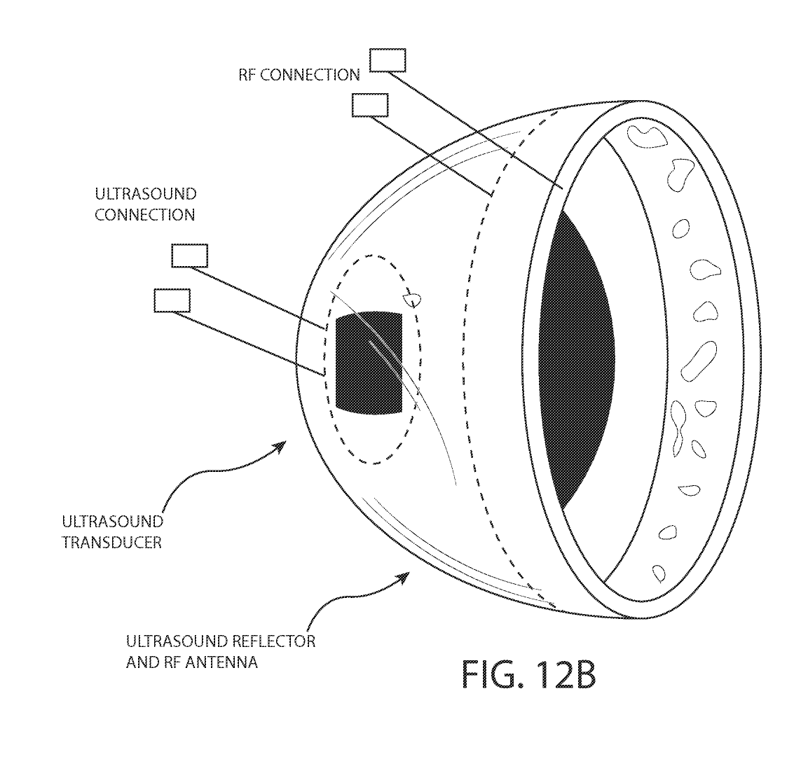

FIGS. 12A and 12B show an example of an ultrasound transducer crystal that can be used to generate an ultrasound field within an environment comprising biological targets. In this particular implementation, the transducer comprises an ultrasound transducer and an RF antenna. The ultrasound transducer further comprises an ultrasound backing layer and a an ultrasound reflector as well as a matching layer. The matching layer in this implementation comprises a fixed matching component and a replaceable, disposable matching component that can protect the transducer from coming into direct contact with the environment, fluids within the environment and/or targets within that environment. In this particular implementation, for example, the transducer is shown generating an electromagnetic signal that includes a first relatively low frequency component of about 200 KHz and a second relatively high frequency component of about 500 Mhz and further generating an ultrasound signal having a frequency range of about 1 to about 14 MHz.

In this implementation the transducer provides a fully integrated ultrasound transducer and an RF antenna element. In some implementations, for example, the ultrasound transducer may comprise a transducer crystal disposed in a back of a reflector. The reflector may include any number of materials, such as a ceramic material. The transducer shown in FIGS. 12A and 12B further include a backing and matching layer that can be made from standard or specially designed materials. Further the waveforms shown represent an interleaved RF and ultrasound delivery. In this implementation, the interleaved timing may can be closely controlled depending upon desired treatment conditions. In an interleaved implementation, for example, the interleaved timing can be used to determine a strategy of thermal or non-thermal discrimination. This enables accurate targeting with a minimal fringe of the RF and ultrasound field, thus enabling laser accuracy targeting but with minimal heating optimization.

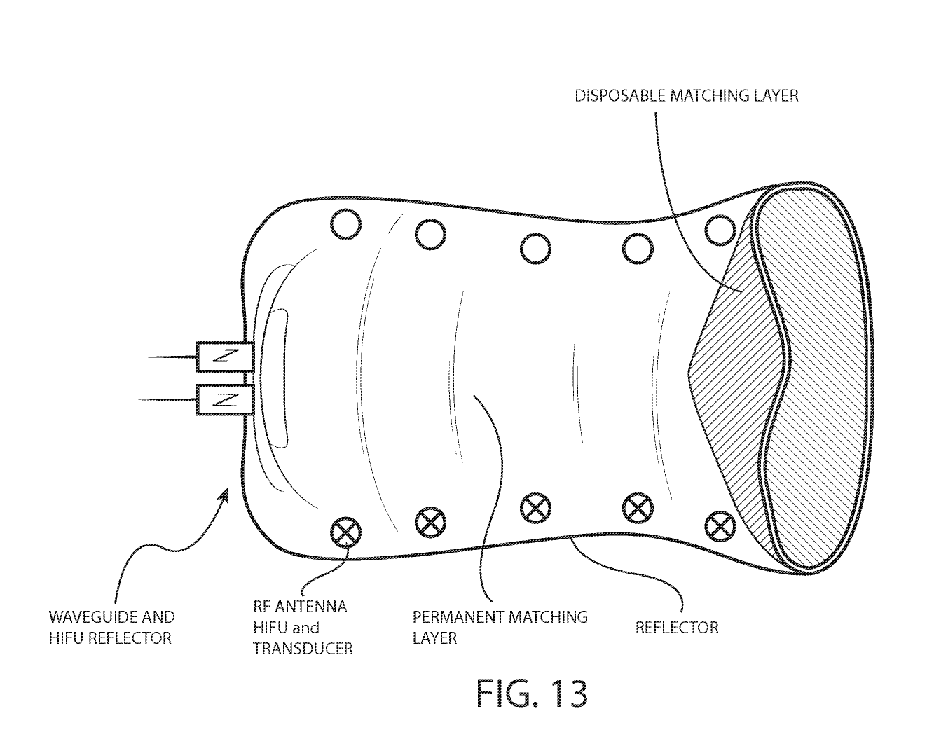

FIG. 13 shows an example transducer including a combination RF antenna and HIFU ultrasound transducer. The ultrasound transducer includes a waveguide and an HIFU reflector for directing the ultrasound energy to the environment. The transducer further comprises a dual matching layer for efficiently transferring energy with the environment. The dual matching layer includes a permanent matching layer and a disposable matching layer than can be removed and discarded after use. In this particular implementation, the RF antenna comprises an external inductive RF coil antenna in which the coil is wound around an exterior of a bell ceramic ultrasound reflector instead of a deposited-sputtered antenna. In this implementation, the coils are represented by dots shown around the exterior of the bell reflector. The transducer is configured to provide an RF field normal to a surface of the environment (e.g., skin) and enable deeper tissue penetration. An ultrasound crystal can be disposed in the back of the bell. In this implementation, the transducer further includes a matching layer designed as a good dielectric as well as a good acoustic matching layer at the same time. The transduce reflector further provides focus for depth of treatment.

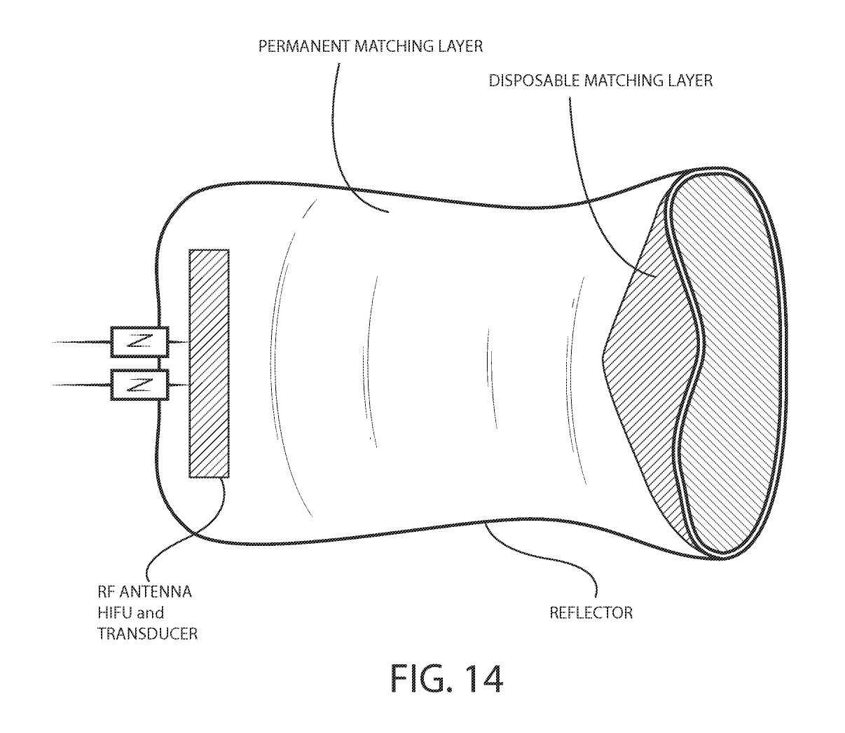

FIG. 14 shows another example transducer for providing electromagnetic and ultrasound excitation to an environment including biological targets. In this particular implementation, the transducer comprises an RF antenna and HIFU ultrasound transducer combination. In this implementation, the antenna has a flat antenna capacitive coupling mesh printed on a flat ultrasound transducer crystal. A ceramic bell reflector also serves a dual purpose as an ultrasound transducer reflector and an RF waveguide. The transducer further comprises disposable matching layers that may be used with an antenna, such as the ones shown in FIGS. 12 and 13, for coupling to the environment while reducing the likelihood that the antenna is contaminated by the biological targets.

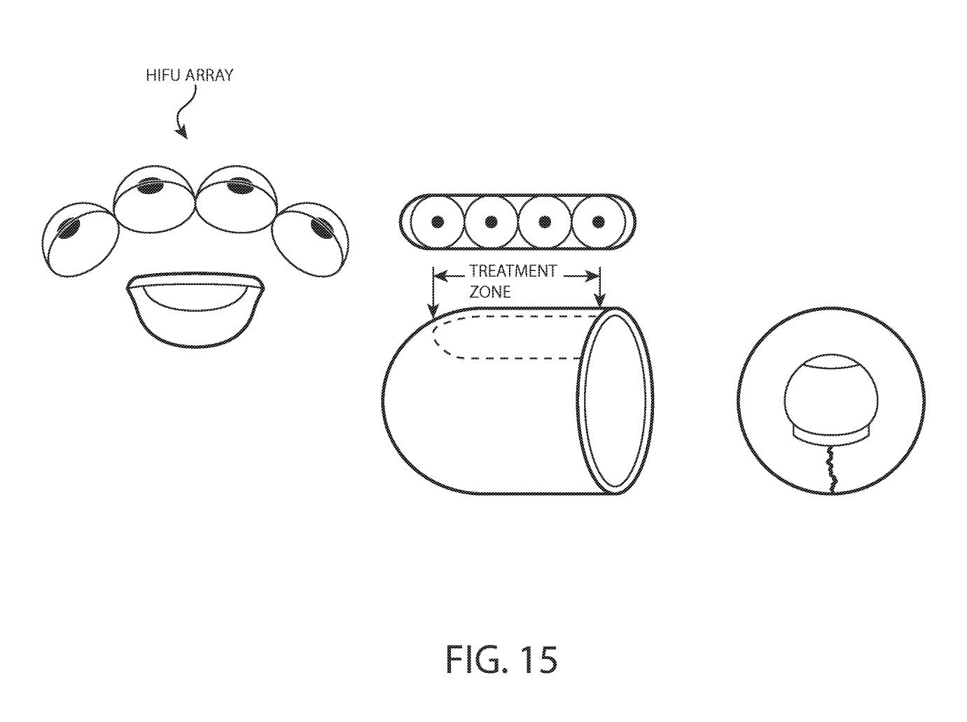

FIG. 15 shows an example of a matching layer or waveguide dielectric comprising an RF inductive antenna coil and an ultrasound transducer that may be used for coupling an electromagnetic field and/or an ultrasound field with an environment comprising biological targets.

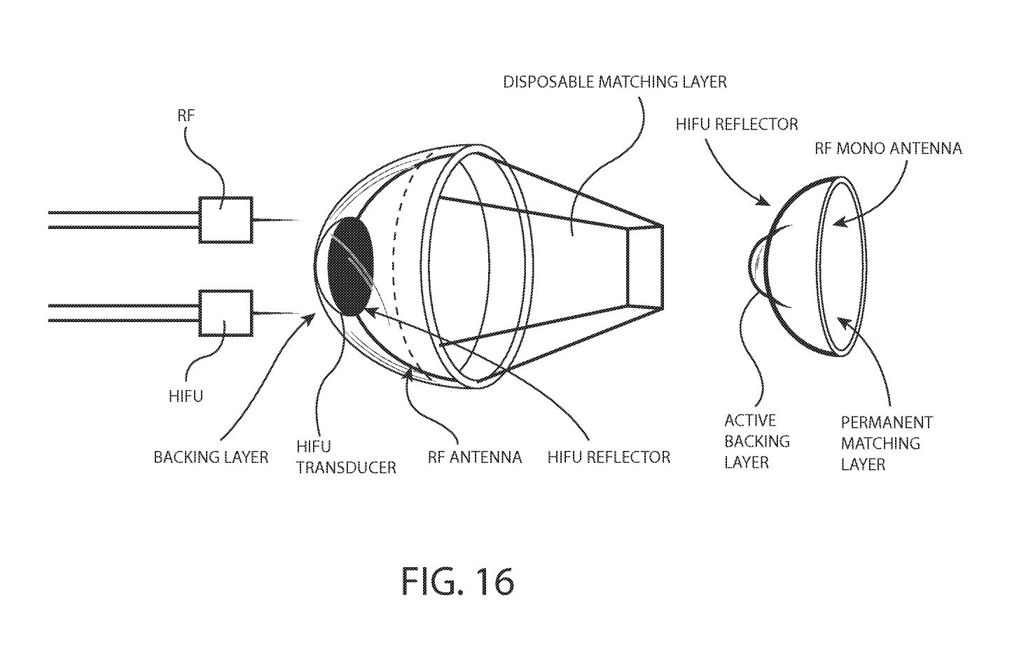

FIG. 16 shows an example of an antenna configured for inductively and capacitively coupling an electromagnetic radio frequency (RF) field and a transducer for coupling an ultrasound field with an environment comprising biological targets. In this implementation, the transducer comprises a HIFU ultrasound transducer including an HIFU reflector, an active backing layer and a permanent matching layer and a disposable matching layer. The transducer further comprises an RF monopole antenna.

The transducer, in this implementation are terminated by the distant matching network to increase or maximize efficiency and for the transducer to be seen by an electronic system as a particular load (e.g., a 50 Ohm load). At a system side, there is a proximal matching network to compensate for a transmission line or handpiece cable. In several of these implementations, such as shown in FIG. 16, the matching layer comprises a unique two part design in which a permanent matching layer (e.g., molded within a dome of a combinational transducer and terminated with a flash surface). The permanent matching layer further includes a connection (e.g., snap or threaded connection) configured to connect with a disposable matching layer.

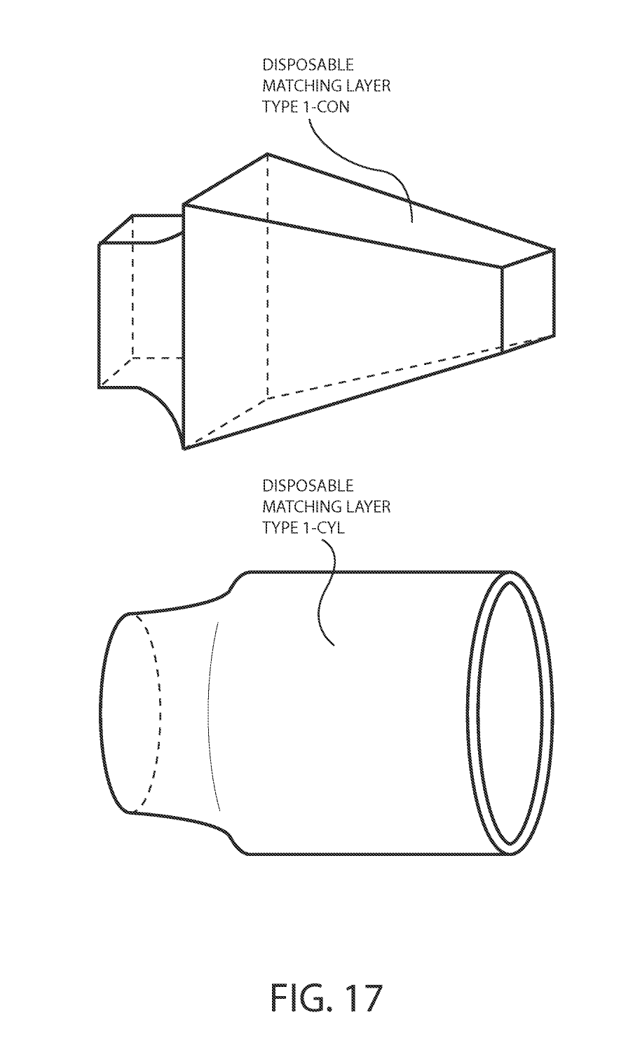

FIG. 17 shows another example of a focused multi-element antenna device for coupling an electromagnetic field and/or ultrasound field with an environment comprising biological targets. In this implementation, two different disposable matching layers are shown for use in an application where the disposable matching layer comes into contact with the environment (e.g., a tissue). In the first implementation, a cylindrical matching layer design provides a shaped structure for matching a first anatomical structure. The second implementation shows a conical design for smaller and more precise areas such as a nail specific area and a larger area designed for a larger surface area treatment such as dermatitis or trauma wounds.

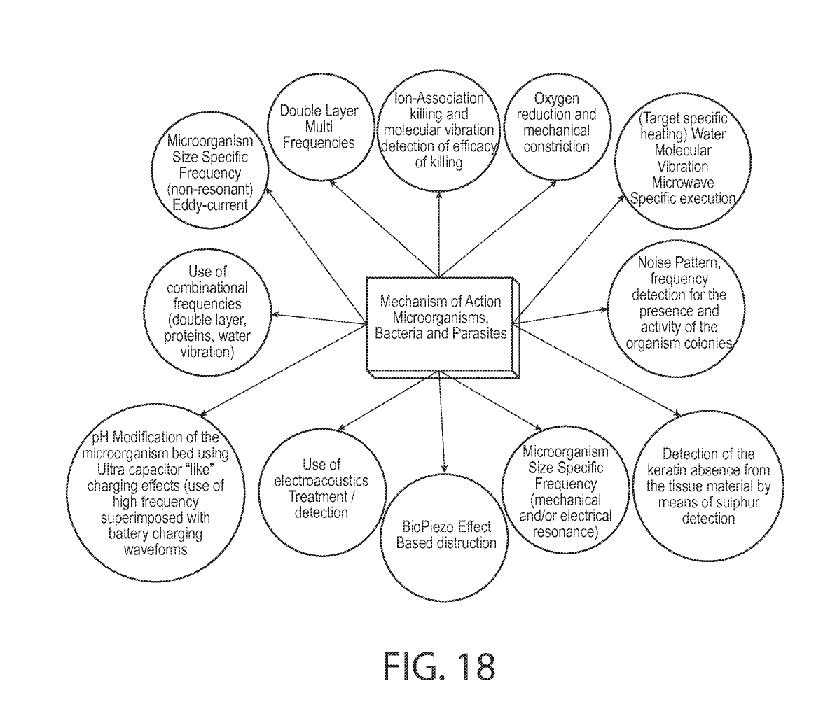

FIG. 18 shows a block diagram of example treatment and detection methodologies that may be used to treat and/or detect biological targets within an environment.

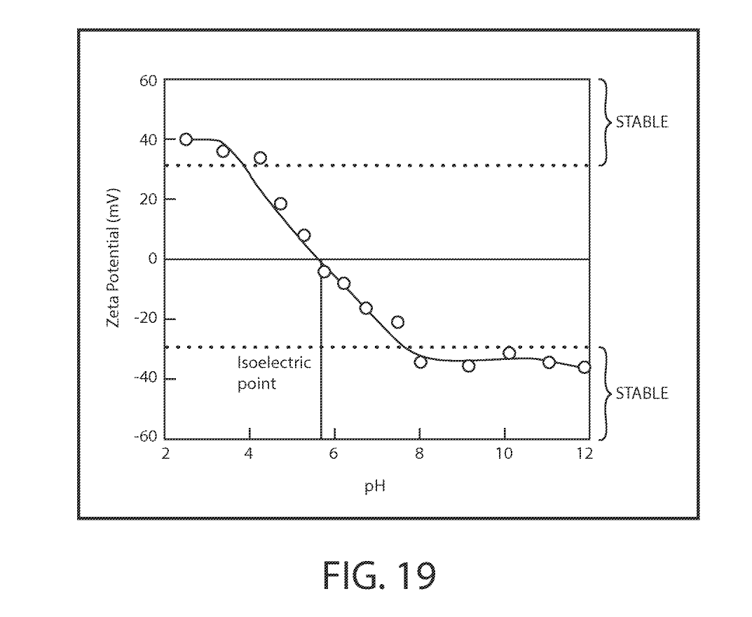

FIG. 19 shows an example graph of a zeta potential plotted with respect to a pH level that may be used in various implementations for detecting biological targets within an environment.

Detection of Biological Targets

In various implementations, one or more techniques of detecting biological targets within an environment can be used to determine the presence, quantity and/or type of biological targets disposed within an environment of interest. The detection of one or more biological target may be performed before treatment to determine whether treatment is warranted, during treatment (e.g., feedback) to determine whether the treatment is working and/or after treatment to determine if the treatment was successful (e.g., determine whether more treatment is warranted or if a different type of treatment is warranted).

Since a pressure wave gradient in an ultrasonic wave moves particles relative to a fluid in which it is located, the motion and the motion of biologic target particles/organisms disturb an electric double layer that exists at a particle-target organism (e.g., a negatively charge ion) interface. The disturbance could come from a motion of live particles/biologic targets as well. Particles carry a surface charge. Ions of a diffuse layer are located in the fluid and can also move with the fluid. Fluid motion relative to the particles can drag these diffuse ions in the direction of one or the other particle's poles. As a result, an excess of negative ions in the vicinity of a left hand pole and an excess of positive surface charge at the right hand pole. This charge excess creates a particle dipole moment. The dipole moments generate an electric field that would generate an electric current. In one implementation, for example, a current is measured, such as via one or more electrodes (e.g., lateral electrodes).

In addition, this data can be used (e.g., indirectly) to calculate a zeta potential in concentrated colloids of the environment. This parameter, in one implementation, is used to measure a degree of repulsion between adjacent particles (e.g., live particles), the activity or degree of infection and/or an efficacy of a treatment (e.g., efficacy of killing and possibly a kill rate). From a theoretical viewpoint, for example, zeta potential is an electric potential in an interfacial double layer (DL) at a location of the slipping plane versus a point in the bulk fluid away from the interface. In other words, zeta potential is the potential difference between a dispersion medium and a stationary layer of fluid attached to a dispersed particle. In one implementation, a flocculation (grouping particles) is created as described above by keeping a zeta potential in real-time in a range from about 0 to about 5 mV. (A typical zeta potential is in mV: (i) from 0 to +5 mV—rapid coagulation or flocculation, (ii) from +/−10 to 30 mV—incipient stability, (iii) +/−30 to 40 mV 000 moderate stability, (iv) from +/−40 to 60—good stability, and (iv) above 61 mV—excellent stability. By electrically charging a treatment zone, a zeta potential may be steered towards flocculation of target particles and thus reduce or even eliminate colonies of biological target particles.

Detection using an electric sonic amplitude is a reverse to colloidal vibration current. Under an influence of an electric field described above with respect to an oscillating electric field, the particles move relative to a liquid which generates ultrasound. In one implementation, an ultrasound echo could be used as a control mechanism. Thus, electroacoustic sensing, treatment and detection may be interrelated. In addition, a link between pH of the environment and the zeta potential may be used so that zeta potential is used to measure pH and a change in pH. See e.g., FIG. 19.

In one implementation, for example, micro-motion detection of particles (e.g., biological targets) using in-phase and quadrature voltage increase as a result of cell surface coverage is performed via one or more electrodes (e.g., lateral electrodes). In one implementation, particle motion and/or growth or reduction in colonies is observed. Motion sensitivity can be in the nanometer (nm) range. It is further possible to observe effects of high burst shock into an area as well as it could be used for control for electrode contact. In addition, mechanical constriction of a treatment area and observation of a change in blood perfusion may also be used. In one implementation, for example, a reduction of perfusion and an increase in oxygen intake may also provide an opportunity to kill or otherwise treat colonies via contact with oxygen-enriched fluid.

In another implementation, a layered structure of tow-finger comprise a layered structure of tissue materials and can be used to detect a location of target particles and dynamic impedance changes due to the presence of particles. In one design based on impedance motion sensing a lock-in amplifier is used for treatment where one electrode used is gold plated and a counter electrode is a second electrode. Changes in electrical resistance reflect attachment and motion of cells and target particles.

In yet another implementation, Brownian noise is used to detect an efficacy of a treatment as well as an extent of bacteria and other biological target presence at a given location. In one implementation, Brownian noise (also referred to as “red noise” refers to a power density that decreases 6 dB per octave with increasing frequency (density proportional to 1/f²) over a frequency range that does not include DC (and in a general sense does not include a constant component, or value at zero frequency). In other implementations, Brownian or “red” noise may refer to any system where power density decreases with increasing frequency. In the firs implementation, Brownian noise can be determined by an algorithm that simulates Brownian motion or by integrating white noise. Brownian noise is not named for a power spectrum that suggests the color brown; rather, the name is related to Brownian motion. “Red noise” describes the shape of the power spectrum, with pink being between red and white. Also known as “random walk” or “drunkard's walk” noise, red noise can be used to detect an efficacy of the treatment as well as the extent of bacteria (or other biological targets) presence at a given location.

In another implementation, dynamic impedance may be used to detect the treatment efficacy and control of the treatment delivery (e.g., as feedback) for any of the aforementioned modalities. A zeta potential (as described above) and/or sulfur detection (e.g., as an indication of a fungus presence) can be used to detect an efficacy of treatment. (See e.g., FIG. 18.)

The embodiments of the invention described herein are implemented as logical steps in one or more computer systems. The logical operations of the present invention are implemented (1) as a sequence of processor-implemented steps executing in one or more computer systems and (2) as interconnected machine or circuit modules within one or more computer systems. The implementation is a matter of choice, dependent on the performance requirements of the computer system implementing the invention. Accordingly, the logical operations making up the embodiments of the invention described herein are referred to variously as operations, steps, objects, or modules. Furthermore, it should be understood that logical operations may be performed in any order, unless explicitly claimed otherwise or a specific order is inherently necessitated by the claim language.

Although embodiments of this invention have been described above with a certain degree of particularity, those skilled in the art could make numerous alterations to the disclosed embodiments without departing from the spirit or scope of this invention. For example, although examples of specific biological targets such as fungus, nail fungus, bacteria, and the like are disclosed in specific examples, one of ordinary skill in the art would recognize from the teaching herein that the apparatuses, systems and methods for treating and/or detecting those biological targets may be altered to treat and/or detect any other number of biological targets in many other types of environments. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims. The above specification, examples, and data provide a complete description of the structure and use of exemplary embodiments of the invention. Since many embodiments of the invention can be made without departing from the spirit and scope of the invention, the invention resides in the claims hereinafter appended. Furthermore, structural features of the different embodiments may be combined in yet another embodiment without departing from the recited claims.

1. A treatment device comprising:
an antenna comprising at least one electrode configured for electrical coupling with an environment comprising biological targets for delivering a first electromagnetic signal to the environment, wherein the first electromagnetic signal comprises a relative low frequency component and a relative high frequency component.

2. The treatment device of claim 1 wherein the low frequency component of the first electromagnetic signal is configured to generate a plurality of ions in the environment.

3. The treatment device of claim 2 wherein the plurality of ions are free to migrate within the environment.

4. The treatment device of claim 2 wherein the plurality of ions are free to migrate within a fluidic suspension of the environment.

5. The treatment device of claim 2 wherein the plurality of ions are generated at a transition adjacent the at least one electrode of the antenna.

6. The treatment device of claim 2 wherein the plurality of ions are generated by an inductive-capacitive resonant charging process via the at least one electrode of the antenna.

7. The treatment device of claim 1 wherein the first electromagnetic signal is configured to generate a plurality of ions in the environment forming an electrical double layer in which a surface charge of the at least one electrode is mirrored by a parallel layer of ions within a fluid of the environment.

8. The treatment device of claim 1 wherein the first electromagnetic signal is configured to generate a plurality of ions in the environment forming an electrical double layer at a transition between the at least one electrode and the environment.

9. The treatment device of claim 1 wherein the first electromagnetic signal is configured to generate a plurality of ions in the environment forming an electrical double layer introduced by an inductive-resonant charging process.

10. The treatment device of claim 1 wherein the first electromagnetic signal is configured to generate a plurality of ions in a fluid of the environment forming a diffuse layer of free ions under an influence of electric attraction.

11. The treatment device of claim 1 wherein the first electromagnetic signal is configured to generate a plurality of ions in a fluid of the environment forming a diffuse layer of free ions under an influence of thermal motion.

12. The treatment device of claim 1 wherein the first electromagnetic signal is configured to generate a plurality of ions in a fluid of the environment forming at a transition within the environment in which different layers or objects have different material or electrical properties.

13. The treatment device of claim 1 wherein the first electromagnetic signal is configured to generate a plurality of ions in a fluid of the environment forming at a tissue transitional surface within the environment.

14. The treatment device of claim 1 wherein the first electromagnetic signal is configured to generate a plurality of ions forming an electrical double layer configured to localize the biological targets.

15. The treatment device of claim 1 wherein the first electromagnetic signal is configured to generate a plurality of ions forming an electrical double layer configured to localize charged colloidal particles of the biological targets.

16. The treatment device of claim 1 further comprising treating the localized biological targets and/or charged colloidal particles of the biological targets.

17. The treatment device of claim 1 further comprising treating the localized biological targets and/or charged colloidal particles of the biological targets in proximity to one another.

18. The treatment device of claim 1 further comprising treating a plurality of coagulated biological target particles or coagulated charged colloidal particles of biological targets.

19. The treatment device of claim 1 further comprising treating a plurality of coagulated biological target particles or coagulated charged colloidal particles of biological targets via one or more of the group comprising bulk heating, a modulated signal, an amplitude modulated radio frequency signal superimposed on a charging direct current signal, electroacoustic energy, electrostatic energy, electrokinetic forces, electrokinetic forces used to compromise a cellular wall of a target particle, pH modification of the environment, pH modification of the environment via ion generation, removing positive ions of a biological target particle or charged colloidal particle of the biological targets.

20. The treatment device of claim 1 wherein a plurality of ions present in the environment that are either generated by the signal or naturally occurring in the environment are free to move within an electric field generated by the first electromagnetic signal.

21. The treatment device of claim 1 wherein the biological targets comprise charged biologic targets.

22. The treatment device of claim 21 wherein the charged biologic targets comprise charged colloidal particles.

23. The treatment device of claim 21 wherein the charged biologic targets comprise charged colloidal particles comprising one or more of the group comprising proteins, bacteria, fungi and biofilm.

24. The treatment device of claim 1 wherein a plurality of ions generated in the environment via the first electromagnet signal are attracted to charged colloidal particles of the biologic targets within the environment.

25. The treatment device of claim 1 wherein a plurality of ions generated in the environment via the first electromagnetic signal surround the charged colloidal particles of the biologic targets within the environment.

26. The treatment device of claim 1 wherein a plurality of ions generated in the environment via the first electromagnetic signal alter a charge of the charged colloidal particles of the biological targets.

27. The treatment device of claim 1 wherein a plurality of ions generated in the environment via the first electromagnetic signal alter a charge of the charged colloidal particles of the biological targets and reduce repulsive forces of the colloidal particles of the biological targets.

28. The treatment device of claim 1 wherein a plurality of ions generated in the environment via the first electromagnetic signal alter a charge of the charged colloidal particles of the biological targets and reduce repulsive forces of the colloidal particles of the biological targets causing the colloidal particles to clot or precipitate within the environment.

29. The treatment device of claim 1 wherein a plurality of ions generated in the environment via the first electromagnetic signal alter a charge of the charged colloidal particles of the biological targets and reduce repulsive forces of the colloidal particles of the biological targets causing the colloidal particles to fall out of solution within the environment.

30. The treatment device of claim 1 wherein the biological targets comprise charged particles and are detected, controlled, oriented, displaced, localized, and/or treated through a capacitive or inductive electrical coupling.

31. The treatment device of claim 1 wherein the first electromagnetic signal applied to the at least one electrode couples the at least one electrode to the environment to provide electrostatic or electromagnetic charge in the environment comprising the biological targets.

32. The treatment device of claim 1 wherein the first electromechanical signal applied to the at least one electrode couples the at least one electrode to the environment to provide electrostatic or electromagnetic charge in the environment comprising charged colloidal particles of the biological targets.

33. The treatment device of claim 32 wherein the biological targets comprise uncharged biological targets.

34. The treatment device of claim 32 wherein the uncharged biologic targets comprise uncharged colloidal particles.

35. The treatment device of claim 32 wherein the environment comprises a fluidic suspension.

36. The treatment device of claim 32 wherein the environment comprises a fluidic suspension of a physiologic system.

37. The treatment device of claim 32 wherein the environment comprises a tissue viscoelastic fluidic suspension.

38. The treatment device of claim 1 wherein the relative low frequency component of the first electromagnetic signal comprises a frequency of less than about 500 KHz.

39. The treatment device of claim 1 wherein the relative low frequency component comprises a frequency in a range from about 5 KHz to about 200 KHz.

40. The treatment device of claim 1 wherein the relative low frequency component comprises a frequency range from about 5 KHz to about 10 MHz.

41. The treatment device of claim 1 wherein the relative high frequency component of the first electromagnetic signal comprises a frequency in the range from 500 MHz to 76 GHz.

42. The treatment device of claim 1 wherein the at least one electrode is in direct contact with a fluid of the environment.

43. The treatment device of claim 1 wherein the at least one electrode is indirectly coupled to the environment.

44. The treatment device of claim 1 wherein the at least one electrode is indirectly coupled to the environment via one or more of air, gel, conductive gel or another separator.

45. The treatment device of claim 1 wherein a plurality of ions generated in the environment alter a pH of the environment.

46. The treatment device of claim 1 wherein a plurality of ions generated in the environment alter a pH of the environment in situ.

47. The treatment device of claim 1 wherein a plurality of ions generated in the environment alter a pH of the environment under control of the first electromagnetic signal.

48. The treatment device of claim 1 wherein a plurality of ions generated in the environment alter a pH of the environment under control of the first electromagnetic signal to create an environment inhospitable to at least one biological target within the environment.

49. The treatment device of claim 1 wherein a plurality of ions generated in the environment alter a pH of the environment under control of the first electromagnetic signal to make the environment more acidic.

50. The treatment device of claim 1 wherein a plurality of ions generated in the environment alter a pH of the environment under control of the first electromagnetic signal to make the environment more basic.

51. A method comprising:
coupling at least one electrode with an environment comprising biological targets;
delivering a first electromagnetic signal to the environment, wherein the first electromagnetic signal comprises a relative low frequency component and a relative high frequency component.

52. The method of claim 51 wherein the low frequency component of the first electromagnetic signal is configured to generate a plurality of ions in the environment.

53. The method of claim 52 wherein the plurality of ions are free to migrate within the environment.

54. The method of claim 52 wherein the plurality of ions are free to migrate within a fluidic suspension of the environment.

55. The method of claim 52 wherein the plurality of ions are generated at a transition adjacent the at least one electrode of the antenna.

56. The method of claim 52 wherein the plurality of ions are generated by an inductive-capacitive resonant charging process via the at least one electrode of the antenna.

57. The method of claim 51 wherein the first electromagnetic signal is configured to generate a plurality of ions in the environment forming an electrical double layer in which a surface charge of the at least one electrode is mirrored by a parallel layer of ions within a fluid of the environment.

58. The method of claim 51 wherein the first electromagnetic signal is configured to generate a plurality of ions in the environment forming an electrical double layer at a transition between the at least one electrode and the environment.

59. The method of claim 51 wherein the first electromagnetic signal is configured to generate a plurality of ions in the environment forming an electrical double layer introduced by an inductive-resonant charging process.

60. The method of claim 51 wherein the first electromagnetic signal is configured to generate a plurality of ions in a fluid of the environment forming a diffuse layer of free ions under an influence of electric attraction.

61. The method of claim 51 wherein the first electromagnetic signal is configured to generate a plurality of ions in a fluid of the environment forming a diffuse layer of free ions under an influence of thermal motion.

62. The method of claim 51 wherein the first electromagnetic signal is configured to generate a plurality of ions in a fluid of the environment forming at a transition within the environment in which different layers or objects have different material or electrical properties.

63. The method of claim 51 wherein the first electromagnetic signal is configured to generate a plurality of ions in a fluid of the environment forming at a tissue transitional surface within the environment.

64. The method of claim 51 wherein the first electromagnetic signal is configured to generate a plurality of ions forming an electrical double layer configured to localize the biological targets.

65. The method of claim 51 wherein the first electromagnetic signal is configured to generate a plurality of ions forming an electrical double layer configured to localize charged colloidal particles of the biological targets.

66. The method of claim 51 further comprising treating the localized biological targets and/or charged colloidal particles of the biological targets.

67. The method of claim 51 further comprising treating the localized biological targets and/or charged colloidal particles of the biological targets in proximity to one another.

68. The method of claim 51 further comprising treating a plurality of coagulated biological target particles or coagulated charged colloidal particles of biological targets.

69. The method of claim 51 further comprising treating a plurality of coagulated biological target particles or coagulated charged colloidal particles of biological targets via one or more of the group comprising bulk heating, a modulated signal, an amplitude modulated radio frequency signal superimposed on a charging direct current signal, electroacoustic energy, electrostatic energy, electrokinetic forces, electrokinetic forces used to compromise a cellular wall of a target particle, pH modification of the environment, pH modification of the environment via ion generation, removing positive ions of a biological target particle or charged colloidal particle of the biological targets.

70. The method of claim 51 wherein a plurality of ions present in the environment that are either generated by the signal or naturally occurring in the environment are free to move within an electric field generated by the first electromagnetic signal.

71. The method of claim 51 wherein the biological targets comprise charged biologic targets.

72. The method of claim 71 wherein the charged biologic targets comprise charged colloidal particles.

73. The method of claim 71 wherein the charged biologic targets comprise charged colloidal particles comprising one or more of the group comprising proteins, bacteria, fungi and biofilm.

74. The method of claim 51 wherein a plurality of ions generated in the environment via the first electromagnet signal are attracted to charged colloidal particles of the biologic targets within the environment.

75. The method of claim 51 wherein a plurality of ions generated in the environment via the first electromagnetic signal surround the charged colloidal particles of the biologic targets within the environment.

76. The method of claim 51 wherein a plurality of ions generated in the environment via the first electromagnetic signal alter a charge of the charged colloidal particles of the biological targets.

77. The method of claim 51 wherein a plurality of ions generated in the environment via the first electromagnetic signal alter a charge of the charged colloidal particles of the biological targets and reduce repulsive forces of the colloidal particles of the biological targets.

78. The method of claim 51 wherein a plurality of ions generated in the environment via the first electromagnetic signal alter a charge of the charged colloidal particles of the biological targets and reduce repulsive forces of the colloidal particles of the biological targets causing the colloidal particles to clot or precipitate within the environment.

79. The method of claim 51 wherein a plurality of ions generated in the environment via the first electromagnetic signal alter a charge of the charged colloidal particles of the biological targets and reduce repulsive forces of the colloidal particles of the biological targets causing the colloidal particles to fall out of solution within the environment.

80. The method of claim 51 wherein the biological targets comprise charged particles and are detected, controlled, oriented, displaced, localized, and/or treated through a capacitive or inductive electrical coupling.

81. The method of claim 51 wherein the first electromagnetic signal applied to the at least one electrode couples the at least one electrode to the environment to provide electrostatic or electromagnetic charge in the environment comprising the biological targets.

82. The method of claim 51 wherein the first electromechanical signal applied to the at least one electrode couples the at least one electrode to the environment to provide electrostatic or electromagnetic charge in the environment comprising charged colloidal particles of the biological targets.

83. The method of claim 82 wherein the biological targets comprise uncharged biological targets.

84. The method of claim 82 wherein the uncharged biologic targets comprise uncharged colloidal particles.

85. The method of claim 82 wherein the environment comprises a fluidic suspension.

86. The method of claim 82 wherein the environment comprises a fluidic suspension of a physiologic system.

87. The method of claim 82 wherein the environment comprises a tissue viscoelastic fluidic suspension.

88. The method of claim 51 wherein the relative low frequency component of the first electromagnetic signal comprises a frequency of less than about 500 KHz.

89. The method of claim 51 wherein the relative low frequency component comprises a frequency in a range from about 5 KHz to about 200 KHz.

90. The method of claim 51 wherein the relative low frequency component comprises a frequency range from about 5 KHz to about 10 MHz.

91. The method of claim 51 wherein the relative high frequency component of the first electromagnetic signal comprises a frequency in the range from 500 MHz to 76 GHz.

92. The method of claim 51 wherein the at least one electrode is in direct contact with a fluid of the environment.

93. The method of claim 51 wherein the at least one electrode is indirectly coupled to the environment.

94. The method of claim 51 wherein the at least one electrode is indirectly coupled to the environment via one or more of air, gel, conductive gel or another separator.

95. The method of claim 51 wherein a plurality of ions generated in the environment alter a pH of the environment.

96. The method of claim 51 wherein a plurality of ions generated in the environment alter a pH of the environment in situ.

97. The method of claim 51 wherein a plurality of ions generated in the environment alter a pH of the environment under control of the first electromagnetic signal.

98. The method of claim 51 wherein a plurality of ions generated in the environment alter a pH of the environment under control of the first electromagnetic signal to create an environment inhospitable to at least one biological target within the environment.

99. The method of claim 51 wherein a plurality of ions generated in the environment alter a pH of the environment under control of the first electromagnetic signal to make the environment more acidic.

100. The method of claim 51 wherein a plurality of ions generated in the environment alter a pH of the environment under control of the first electromagnetic signal to make the environment more basic.

101. The method of claim 51 wherein the relative low frequency component comprises a frequency below about 1 MHz and the relative high frequency component comprises a frequency in a range from about 100 MHz to about 220 GHz.

102. The method of claim 51 wherein the biologic target comprises a biofilm and the environment comprises a substrate.

103. The method of claim 51 wherein the relative low frequency component comprises a frequency less than about 1 MHz.

104. The method of claim 51 wherein the relative high frequency component of the first electromagnetic signal comprises a frequency in the range from about 1 MHz to about 220 GHz.

105. The treatment device of claim 1 wherein the relative low frequency component comprises a frequency below about 1 MHz and the relative high frequency component comprises a frequency in a range from about 100 MHz to about 220 GHz.

106. The treatment device of claim 1 wherein the biologic target comprises a biofilm and the environment comprises a substrate.

107. The treatment device of claim 1 wherein the relative low frequency component comprises a frequency less than about 1 MHz.

108. The treatment device of claim 1 wherein the relative high frequency component of the first electromagnetic signal comprises a frequency in the range from about 1 MHz to about 220 GHz.

109. A method of treating a biological target within an environment, the method comprising:
coupling at least one electrode with an environment comprising biological targets;
delivering a first electromagnetic signal to the environment, wherein the first electromagnetic signal comprises a relative low frequency component and a relative high frequency component;
performing at least one of the group comprising: (a) directly or indirectly electrically couples to physiological fluids in the infected environment to introduce ions into the fluid; (b) migrate ions, including certain biologic targets, within the infected environment into a location where they can be more easily treated and/or detected; (c) locally alter a pH of the infected environment through an introduction or movement of ions to treat the biologic targets; (d) use the introduced ions to reduce repulsive forces of the biologic targets within the physiological fluid and cause the targets to clot or precipitate within a region of the infected environment; and (e) once localized, treat the biologic targets.

110. The method of claim 109 wherein the operation of, once localized treat the biologic targets comprises at least one of the group comprising: (i) treat the biologic targets or more of the through bulk heating, a modulated signal, electro-acoustic energy and/or pH modification;
(ii) use electrostatic, electro-acoustic, and/or electro-kinetic forces to treat a biologic target (e.g., use electrostatic, electro-acoustic, and/or electro-kinetic forces to compromise a cell wall of a biologic target compromise a cell wall of a biologic target); (iii) use ultrasound frequencies to cause motion of a complex target-external ion and generate electrical current in the infected environment; and/or (iv) expose the localized biologic targets to broadband light or components of broadband light that are effective in treating the biologic targets.

111. A method of detecting at least one biological targets within an environment, the method comprising using at least one operation of a group of operations to detect the at least one biological target, the group comprising: (i) detecting a current caused by a particle dipole moment; (ii) calculating zeta potential in concentrated colloids to measure a degree of repulsion between adjacent particles; (iii) electrically charging a treated zone to steer a zeta potential to encourage particle flocculation; (iv) using electric sonic amplitude to reverse a colloidal vibration current; (v) use zeta potential to measure pH; (vi) use zeta potential to measure a change in pH; (vii) monitor particle motion; (viii) determine a change in blood perfusion; (ix) detect location of a target particle via a layered tow-finger structure; (x) monitor a dynamic impedance change; (xi) monitor a dynamic impedance change via a lock in amplifier; (xii) determine Brownian motion to detect extent of target presence or efficacy of treatment; (xiii) detect sulfur.

112. The method of claim 111 wherein a result of the operation to detect the at least one biological target is used as feedback in a method to treat the biological targets.
